(19) United States
(12) Patent Application Publication
(10) Pub. No.: US 2023/0041627 A1
(43) Pub. Date: Feb. 9, 2023

(54) ASSAY COMPOUND SCREENING

(71) Applicant: University of Connecticut, Farmington, CT (US)

(72) Inventors: Adam Zweifach, Storrs, CT (US); Haim Bar, Mansfield, CT (US)

(21) Appl. No.: 17/881,387

(22) Filed: Aug. 4, 2022

Related U.S. Application Data
(60) Provisional application No. 63/229,385, filed on Aug. 4, 2021.

(51) Int. Cl. G16C 20/30; G16B 5/20; C12N 15/10

(52) U.S. Cl. CPC G16C 20/30; G16B 5/20; C12N 15/1089

(57) ABSTRACT

A method includes receiving measured activity from a first assay, the first assay comprising wells containing compounds and first controls. The measured activity may be indicative of activation or inhibition by the compounds on a process. The method includes determining an estimate of activity percentages of the compounds. The estimate of the activity percentages of the compounds may be based on a noise distribution, and the noise distribution may be based on the first controls. The method includes receiving a first reference based on a required activity percentage of compounds for the first assay. The method includes receiving a second reference based on a required accuracy percentage of the compounds identified as active according to the first reference. The method includes identifying active compounds of the first assay based on the first reference, the second reference, the measured activity, and the estimate of activity percentages.

### CROSS-REFERENCE TO RELATED APPLICATIONS

This application claims the benefit of U.S. Provisional Application No. 63/229,385, filed Aug. 4, 2021, which is incorporated herein in its entirety.

### BACKGROUND

One triumph of modern medicine is the development of thousands of safe and effective medicines for a wide range of human disease. Throughout history, therapeutic drugs have been identified through prior experiences with plants and other natural sources or by accident. Automated liquid handling has brought the era of large-scale screenings to identify numerous compounds, molecules, or biological candidates for development into drugs or other purposes. This autonomous or semi-autonomous screening or testing is sometimes referred to as high-throughput screening (HTS), which describes the large quantities of compounds that may be tested simultaneously or under similar experimental conditions in an assay. The screening of compounds may generate false positives and false negatives, over-including compounds as active when they are not and under-including compounds as inactive when they are not.

### SUMMARY

The present disclosure relates to the identification, selection, and synthesis of compounds. It is to be understood that both the following general description and the following detailed description provide only examples and are not restrictive.

A method may include receiving measured activity from a first assay, the first assay comprising wells containing compounds and first controls. The measured activity may be indicative of activation or inhibition by the compounds on a process. The method may include determining an estimate of activity percentages of the compounds. The estimate of the activity percentages of the compounds may be based on a noise distribution, and the noise distribution may be based on the first controls. The method may include receiving a first reference based on a required activity percentage of compounds for the first assay. The method may include receiving a second reference based on a required accuracy percentage of the compounds identified as active according to the first reference. The method may include identifying active compounds of the first assay based on the first reference, the second reference, the measured activity, and the estimate of activity percentages.

A method may include synthesizing a compound of compounds. The compound may be selected by steps. The steps may include receiving measured activity from a first assay, the first assay comprising wells containing compounds and first controls. The measured activity may be indicative of activation or inhibition by the compounds on a process. The method may include determining an estimate of activity percentages of the compounds. The estimate of the activity percentages of the compounds may be based on a noise distribution, and the noise distribution may be based on the first controls. The method may include receiving a first reference based on a required activity percentage of compounds for the first assay. The method may include receiving a second reference based on a required accuracy percentage of the compounds identified as active according to the first reference. The method may include identifying active compounds of the first assay based on the first reference, the second reference, the measured activity, and the estimate of activity percentages.

A method may include conducting an experiment on a second assay based on active compounds identified by steps. The steps may include receiving measured activity from a first assay, the first assay comprising wells containing compounds and first controls. The measured activity may be indicative of activation or inhibition by the compounds on a process. The method may include determining an estimate of activity percentages of the compounds. The estimate of the activity percentages of the compounds may be based on a noise distribution, and the noise distribution may be based on the first controls. The method may include receiving a first reference based on a required activity percentage of compounds for the first assay. The method may include receiving a second reference based on a required accuracy percentage of the compounds identified as active according to the first reference. The method may include identifying active compounds of the first assay based on the first reference, the second reference, the measured activity, and the estimate of activity percentages.

### BRIEF DESCRIPTION OF THE DRAWINGS

In order to provide understanding techniques described, the figures provide non-limiting examples in accordance with one or more implementations of the present disclosure, in which:

FIG. 1 illustrates example statistical power distributions based on a screening in accordance with one or more implementations of the present disclosure;

FIG. 2 illustrates the postulated true activity of compounds in accordance with one or more implementations of the present disclosure;

FIG. 3 illustrates observed or simulated activities in accordance with one or more implementations of the present disclosure;

FIG. 4 illustrates an identification of compounds as a function of activity with respect to a variance of 2% in accordance with one or implementations of the present disclosure;

FIG. 5 illustrates an identification of compounds as a function of activity with respect to a variance of 8.5% in accordance with one or implementations of the present disclosure;

FIG. 6 illustrates an identification of compounds as a function of activity with respect to a variance of 13.5% in accordance with one or implementations of the present disclosure;

FIG. 7 illustrates an identification of compounds as a function of activity with respect to a variance of 16.5% in accordance with one or implementations of the present disclosure;

FIG. 8 illustrates an example screening system in accordance with one or more implementations of the present disclosure; and

FIG. 9 illustrates an example method in accordance with one or more implementations of the present disclosure.

### DETAILED DESCRIPTION

It is understood that when combinations, subsets, interactions, groups, etc. of components are described that, while specific reference of each various individual and collective combinations and permutations of these may not be explicitly described, each is specifically contemplated and described herein. This applies to all parts of this application including, but not limited to, steps in described methods. Thus, if there are a variety of additional steps that may be performed it is understood that each of these additional steps may be performed with any specific configuration or combination of configurations of the described methods.

As will be appreciated by one skilled in the art, hardware, software, or a combination of software and hardware may be implemented. Furthermore, a computer program product on a non-transitory computer-readable storage medium having processor-executable instructions (e.g., computer software) embodied in the storage medium. Any suitable computer-readable storage medium may be utilized including hard disks, CD-ROMs, optical storage devices, magnetic storage devices, memresistors, Non-Volatile Random Access Memory (NVRAM), flash memory, or a combination thereof.

Throughout this application reference is made to block diagrams and flowcharts. It will be understood that each block of the block diagrams and flowcharts, and combinations of blocks in the block diagrams and flowcharts, respectively, may be implemented by processor-executable instructions. These processor-executable instructions may be loaded onto a special purpose computer or other programmable data processing instrument to produce a machine, such that the processor-executable instructions which execute on the computer or other programmable data processing instrument create a device for implementing the functions specified in the flowchart block or blocks.

These processor-executable instructions may also be stored in a computer-readable memory or a computer-readable medium that may direct a computer or other programmable data processing instrument to function in a particular manner, such that the processor-executable instructions stored in the computer-readable memory produce an article of manufacture including processor-executable instructions for implementing the function specified in the flowchart block or blocks. The processor-executable instructions may also be loaded onto a computer or other programmable data processing instrument to cause a series of operational steps to be performed on the computer or other programmable instrument to produce a computer-implemented process such that the processor-executable instructions that execute on the computer or other programmable instrument provide steps for implementing the functions specified in the flowchart block or blocks.

Blocks of the block diagrams and flowcharts support combinations of devices for performing the specified functions, combinations of steps for performing the specified functions and program instruction means for performing the specified functions. It will also be understood that each block of the block diagrams and flowcharts, and combinations of blocks in the block diagrams and flowcharts, may be implemented by special purpose hardware-based computer systems that perform the specified functions or steps, or combinations of special purpose hardware and computer instructions.

The method steps recited throughout this disclosure may be combined, omitted, rearranged, or otherwise reorganized with any of the figures presented herein and are not intend to be limited to the four corners of each sheet presented.

A goal of screening may be to find as many compounds as possible that present or exceed a selected level of activity. As an example, this activity may be an inhibition or activation of a test process. Compounds found that do not meet the selected level of activity (e.g., inhibition or activation) in an initial test may be removed from subsequent tests or iterations, identifying compounds desired for additional testing.

Another concept sometimes used in screening is the notion of Type I (false positive) and Type II (false negative) errors occurring while trying to distinguish normally-distributed “inactive” compounds from normally-distributed “active” compounds. Although compounds may be categorized into active and inactive categories, this does not accurately depict the underlying reality in that activity is a continuum or spectrum of activity that ranges from completely inactive to fully active. For instance, compounds may fail to neatly fall into active or hit categories and inactive or non-hit categories.

Metrics, such as Z′-factor, or Z′, are used to measure assay quality. The equation for Z′-factor is provided in Equation 1.

$${Z}^{′} = 1 - \frac{3 * ( {σ}_{pc} + {σ}_{nc} )}{\vert {μ}_{p c} - {μ}_{n c} \vert} \qquad ( 1 )$$

where σ and μ represent standard deviation and mean, respectively, of normalized positive (pc) and negative (nc) controls that can be included in assay plates along with test compounds.

Typically, the Z′-factor reflects noise or errors introduced into assay measurements that are indicative of the sum of the errors arising from the underlying biology, the complex liquid and sampling handling, and precision with which compounds can be dispensed into wells, and the underlying noise characteristics of the measurement instrumentation. These errors are quantified by the standard deviation (o) of the signal, measured at a particular level. In some cases, the errors are normally distributed, while in others they are not. Because Z′-factor is determined from normalized signal amplitudes, it is dependent on a.

In an example, 40,000 compounds may be tested by a screening process where half of the compounds are entirely inactive with 0% inhibition and the other half are active with varying levels of inhibition. These compounds can be binned into a quantity of bins (e.g., 50) with a quantity of active compounds in each bin.

From controls setup by the assayer, σ_{pc} and σ_{nc} may be estimated, and these will reflect noise that will be introduced to the results. As an example, the noise may be Gaussian, and the noise may have a standard deviation based on the σ_{pc}, σ_{nc} or combinations thereof. The introduced noise may be used to further identify the actual activity or inactivity of the compounds beyond what is predicted with the original Z′-factor alone.

In some cases, measurements will indicate that a constant deviation may be assumed to analyze the data. For instance, if

$$\frac{{σ}_{pc}}{{σ}_{nc}} = 1 \qquad ( 2 )$$

$$then$$

$${σ}_{experiment} = \frac{( 1 - {Z}^{′} )}{6} \qquad ( 3 )$$

In some cases, the standard deviation may be measured to be proportional to the detected inhibition in the experiment (e.g., 50%). In such a way, the average standard deviation is determined by the assays Z′ and may be equal to σₑₓₚₑᵣᵢₘₑₙₜ

$${σ}_{experiment} ( p ) = \frac{2 {σ}_{0}}{C + 1} * ( C + \frac{p ( 1 - C )}{100} ) \qquad ( 4 )$$

where p is the percent of inhibition or activation and C is a constant. σₑₓₚₑᵣᵢₘₑₙₜ may be a linear function of the percent of inhibition and σ₀ is defined in Equation 5.

$${σ}_{0} = \frac{1}{2} ( {σ}_{0} ( 100 ) + {σ}_{0} ( 0 ) ) \qquad ( 5 )$$

For example, if Z′=0.4, σ₀ may be equal to 0.1 and if C=5, σ₀(100)=0.2/6 and σ₀(0)=⅙. For C=∞, σₑₓₚₑᵣᵢₘₑₙₜ(p) is shown as Equation 6.

$${σ}_{experiment} ( p ) = \frac{2 {σ}_{0} ( 100 - p )}{100} \qquad ( 6 )$$

In such a way, the standard deviation of the experiment may be determined based on the activation or inhibition percentage of the experiment, and the imparted noise added to the distribution may match the standard deviation.

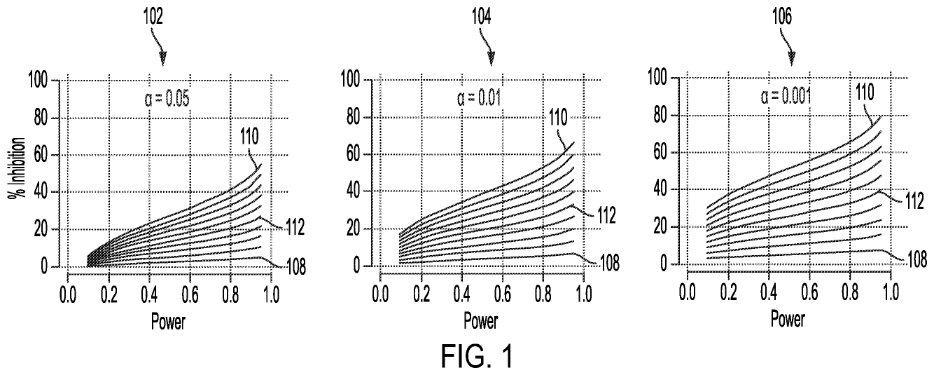

In FIG. 1, example power distributions 102, 104, 106 based on a screening in accordance with one or more implementations of the present disclosure is shown. One way to assess the effect of Z′ on assay performance is to estimate power (e.g., 1−β), where β is the Type II error rate. In other words, if β is the error rate of false negatives, power may be similar to the correct prediction rate of true positives (e.g., activations). α may be the error rate of false positives (e.g., Type I), and setting the false positives constant may present indications of inhibition over power as shown in the example power distributions 102, 104, 106. For instance, power of the experiment may be predicted by controlling the Type I error rate, and techniques disclosed herein may predict a power of the experiment while controlling the Type I error rate. As an example, power distribution 102 depicts how power depends on inhibition with a false positive error rate of 5%, power distribution 104 depicts inhibition over power with a false positive error rate of 1%, and power distribution 106 depicts inhibition over power with a false positive error rate of 0.1%. As discussed herein, if the error rates for Type I and Type II errors are known, additional insight into the actual inhibition percentages may be determined, providing compounds that may be indicative of hits that are associated with a Z′-factor less than 0.5 (e.g., 0.1, 0.2, 0.3, 0.4). Each curve in the example power distributions 102, 104, 106 relates to Z′-factors between zero (e.g., Z′-factor 108) and 0.9 (e.g., Z′-factor 110) incremented by 0.1, Z′-factor 112 is indicative of a 0.4 Z′-factor.

The example power distributions may be provided according to Equation 7.

$$power = {\int }_{- ∞}^{{C}_{a}} \frac{1}{\sqrt{2 πσ}} exp ( - \frac{{( x - μ )}^{2}}{2 {σ}^{2}} ) dx , \qquad ( 7 )$$

where μ<1 is the activity under the alternative, and the one-sided Cₐ cutoff controls the probability of a Type I error at the a level under the null hypothesis of no activity and mean equal to one as shown in Equation 8.

$$power = {\int }_{- ∞}^{{C}_{a}} \frac{1}{\sqrt{2 π σ}} exp ( - \frac{{( x - 1 )}^{2}}{2 {σ}^{2}} ) dx \qquad ( 8 )$$

As an example, although an assay with Z′-factor of 0.4 (e.g., Z′-factor 112) shown in power distribution 106 is less than the industry standard requirement of Z′-factors greater than 0.5, power analysis indicates that the assay can reliably find compounds that inhibit by˜40%. As an example, an assay with a Z′-factor of 0.5 may reach 80% power when compounds inhibit by greater than 20%. Assays with a Z′-factor less than 0.5 (e.g., Z′=0.1) may reach 80% power when inhibition is greater than 36%. For α<0.001, as shown in power distribution 106, which also corresponds to the greater than three standard deviations assumption that is implicit in the definition of Z′-factors, an assay with a Z′-factor of 0.9 reaches 80% power for compounds that inhibit by greater than 6.7%. As an example, a Z′-factor of 0.1 reaches 80% power when inhibition is greater than 58%. For this reason, assays with Z′-factors below 0.5 during the assay development phase or during the primary screening may still be used.

When analyzing assay performance, it may be insufficient to learn how many active compounds assays with different Z′-factors will find, it may also be desirable to know how active those compounds are likely to be. For instance, not only the mere presence of any inhibition or activation, but further prediction of the strength of inhibition or activation. It may also be of interest to estimate how many compounds with a given level of activity will be missed. Because of the noise, reflected in the σₑₓₚₑᵣᵢₘₑₙₜ(P) as described above, the effect of a compound measured in an assay may not be the true value of its effect. Instead, the true effect of a given compound may lie probabilistically within a normal distribution with width defined by the standard deviation that includes the measured value.

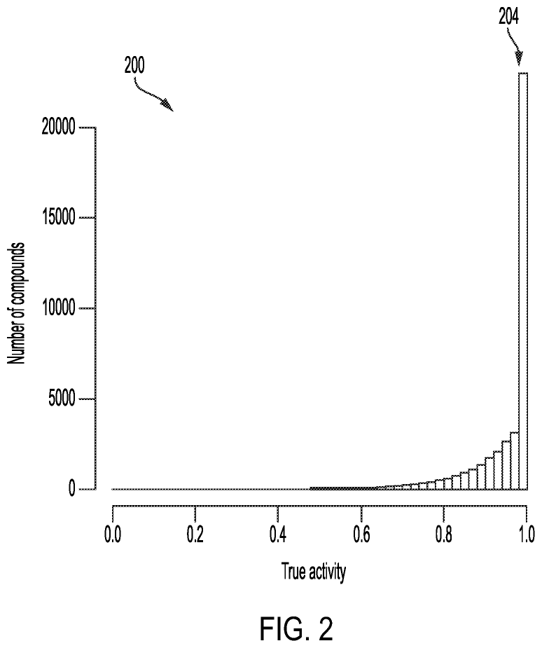

In FIG. 2, the postulated true activity 204 of compounds in accordance with one or more implementations of the present disclosure is shown in a chart. As an example, the postulated true activity 204 may be an inhibition percentage. The postulated true activity may be separated into bins as shown. Put another way, the postulated true activity 204 may be provided or postulated as an assumption by a practitioner based on knowledge and skill in the art. The postulated true activity 204 may be estimated or derived as an activation or inhibition percentage or a confidence of activity.

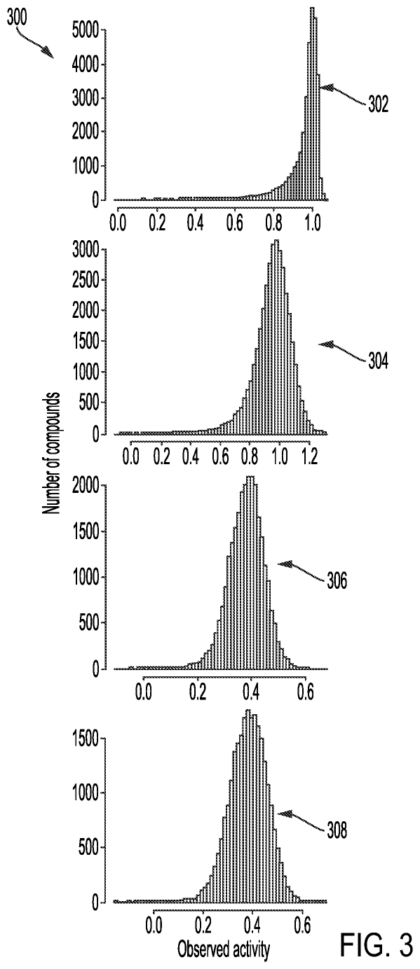

For example, the postulated true activity 204 may be an estimation of the true activity based on library values or different estimated distributions of activations or inhibitions. For example, the library values may form a set of values based on previous experiments or gathered from various databases. The postulated true activity 204 may be generated using the distributions of apparent compound activities that are generated from input compound activity and error distributions. The estimation may be based on curve fitting for the library of activation or inhibition data. For example, generic compound distributions may be used as seeds for curve fitting. Curve-fitting algorithms may include methods and processes that quantitatively analyze goodness-of-fit, reducing error between the set of measured activity percentages and the estimate of the activity percentages from the first assay. The methods and processes may be iterative. For example, R packages may be used (e.g., twosamples) to iteratively estimate and best-fit models of the library compound activity. Some data sets may include compounds that have been measured enough times to have a good estimate of true activity, acting as a seed for curve fitting and estimation. The range of values may be obtained through averaging underlying models or parameters to best estimate the postulated true activity. The estimate may be unique or pseudo-unique for each user or related to a user identity and may unique or pseudo-unique for each assay. The estimate may predict the underlying compound distribution model or models. The curve fitting may be an iterative process or a repeated sequence of steps that reduces the error between FIG. 2 and FIG. 3 until a predetermined value of error is identified.

In FIG. 3, the observed activities in accordance with one or more implementations of the present disclosure are shown. As examples, the observed activity 302 is shown with a Z′-factor of 0.9. The observed activity 304 is shown with a Z′-factor of 0.5. The observed activity 306 is shown with a Z′-factor of 0.2. The observed activity 308 is shown with a Z′-factor of zero. Other Z′-factors are contemplated by this disclosure. Each Z′-factor is determined from estimates of σₑₓₚₑᵣᵢₘₑₙₜ(p) (e.g., postulated true activity 204) and is related to a variance as described.

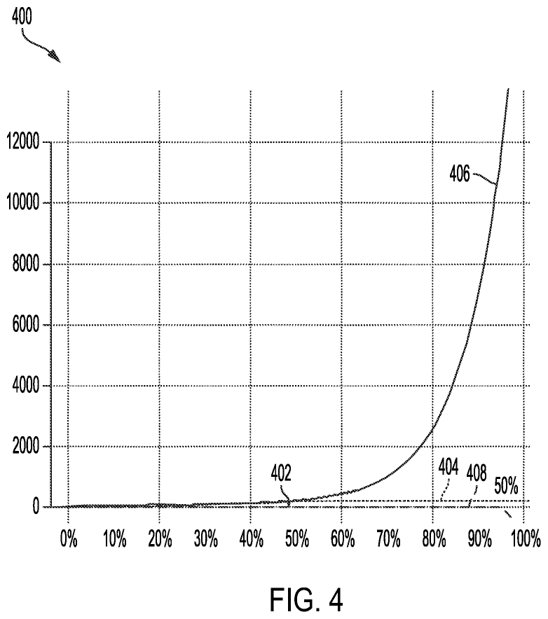

In FIG. 4, an identification of compounds as a function of activity with respect to a variance of 2% (Z′ factor of 0.9, with constant noise across the activity spectrum) in accordance with one or implementations of the present disclosure. An activity percentage 404 is shown where the total number of compounds found activate or inhibit 50% or more. The accuracy percentage 402 is indicative of where 80% of all compounds identified inhibit at the activity percentage 404. Values for the accuracy percentage 402 may be input by a user, required by an algorithm, adjusted based on error or an activity percentage, or combinations thereof. Line 406 is indicative of the total number of compounds found, and line 408 is indicative of the quantity of totally inactive compounds that are erroneously identified as active.

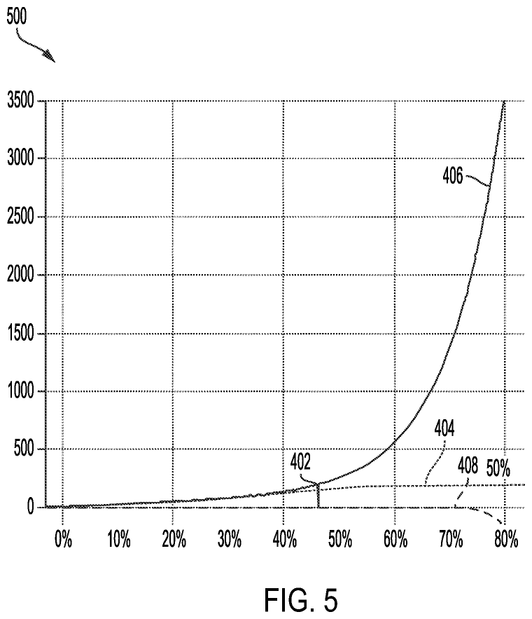

In FIG. 5, an identification of compounds as a function of activity with respect to a variance of 8.5% (Z′=0.5, with constant noise across the activity spectrum) in accordance with one or implementations of the present disclosure. An activity percentage 404 is shown where the total number of compounds found inhibit 50% or more. The accuracy percentage 402 is indicative of where 80% of all compounds identified inhibit at the activity percentage 404. Line 406 is indicative of the total number of compounds found, and line 408 is indicative of the quantity of totally inactive compounds that are erroneously identified as active.

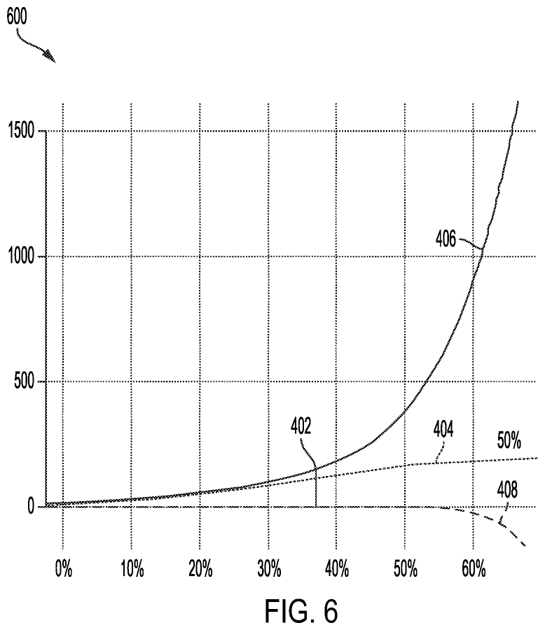

In FIG. 6, an identification of compounds as a function of activity with respect to a variance of 13.5% (Z′=0.2, with constant noise across the activity spectrum) in accordance with one or implementations of the present disclosure. An activity percentage 404 is shown where the total number of compounds found inhibit 50% or more. The accuracy percentage 402 is indicative of where 80% of all compounds identified inhibit at the activity percentage 404. Line 406 is indicative of the total number of compounds found, and line 408 is indicative of the quantity of totally inactive compounds that are erroneously identified as active.

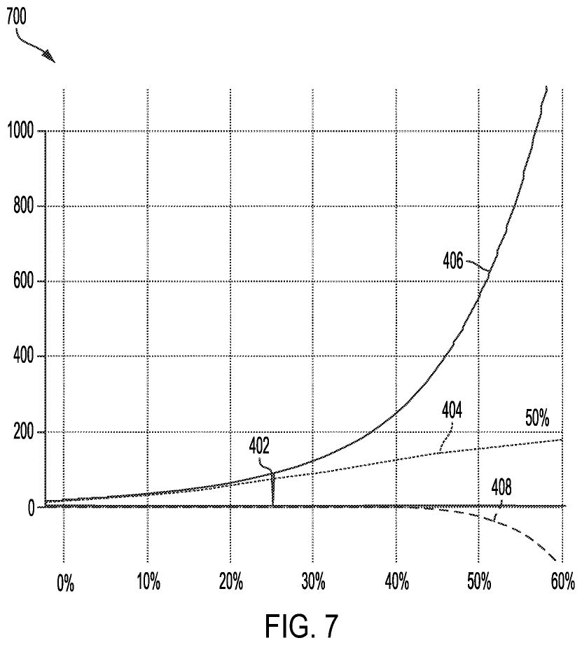

In FIG. 7, illustrates an identification of compounds as a function of activity with respect to a variance of 16.5% (Z′=0, with constant noise across the activity spectrum) in accordance with one or implementations of the present disclosure. An activity percentage 404 is shown where the total number of compounds found inhibit 50% or more. The accuracy percentage 402 is indicative of where 80% of all compounds identified inhibit at the activity percentage 404. Line 406 is indicative of the total number of compounds found, and line 408 is indicative of the quantity of totally inactive compounds that are erroneously identified as active.

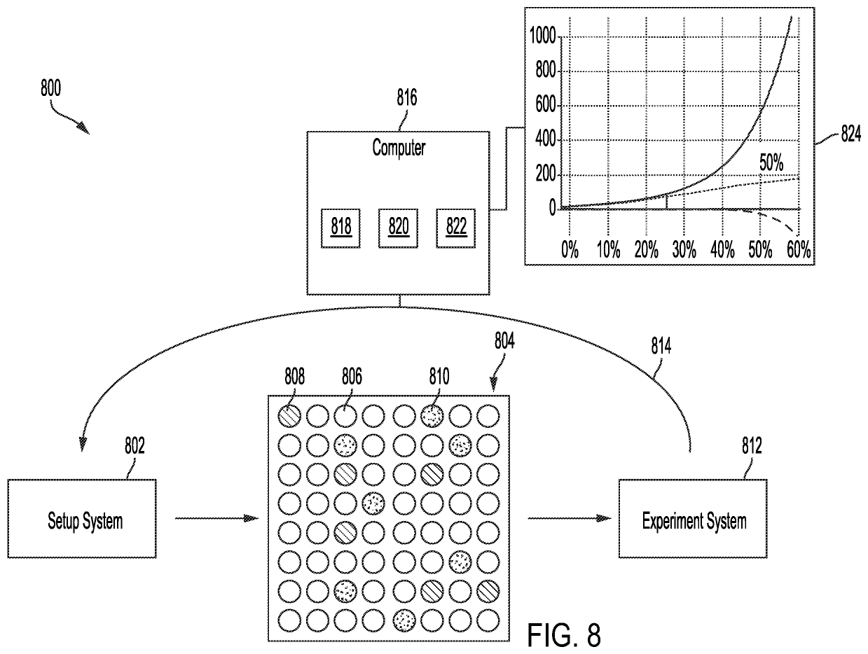

In FIG. 8, an example screening system 800 in accordance with one or more implementations of the present disclosure is shown. The system 800 may include a setup system 802. As an example, the setup system 802 may fill an assay of wells 804. The setup system 802 may autonomously or semi-autonomously fill the assay of wells 804. The wells 804 may be filled with test compounds 806 to test a hypothesis or perform an experiment on the test compounds. In order to determine experiment noise or a Z′-factor, additional wells 804 may be filled with a positive control 808 and a negative control 810. A negative control group of negative controls 810 may be a control group that is not exposed to the experimental treatment or to any other treatment that is expected to have an effect. A positive control group of positive controls 808 may be a control group that is not exposed to the experimental treatment but that is exposed to some other treatment that is known to produce the expected effect.

The assay of wells 804 may be configured and processed for experimentation in an experiment system 812. The experiment system 812 may autonomously or semi-autonomously conduct an experiment on the assay of wells 804. As such, results 814 are provided to a computer 816. The computer 816 may be configured to receive the results 812. In an example, the computer 816 may also be configured to operate or actuate the setup system 802 and the experiment system 812. In an example, the computer 816, or apparatus, may include a network interface 818, a computer-readable medium 820, and a processor 822. The network controller 818 may be configured to communicated with other systems for evaluating the results 814 or provide access to a user. A user may review the results 814 and perform a secondary screening based on the results 814. As an example, the computer 816 may conduct a secondary screen with active compounds identified with methods defined herein. The computer 816 may include a display 824 for indicating the accuracy percentage 402 and activity percentage 404.

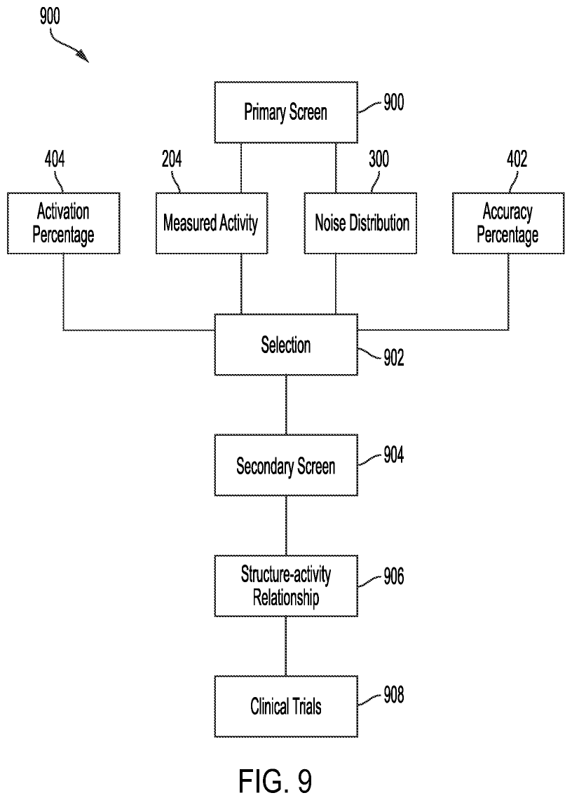

In FIG. 9, an example method 900 in accordance with one or more implementations of the present disclosure is shown. The method 900 may be performed by one or more systems, apparatuses, processors, memories, or combinations thereof. The method begins in step 902 with a primary screen. The primary screen may test an assay for inhibitions or activations for a variety of compounds. In an example, the primary screen may be pulled from a database repository of primary screens by computer 816 over network interface 818. The computer 816 or another implement may be configured to receive the activity percentage 404, the accuracy percentage 402, the measured activity 204, and the noise distribution 300. The activity percentage 404 may be an input from a user. The accuracy percentage 402 may be an input from a user or determined as described herein. The display 824 may be used to indicate the identification of compounds depicted in FIGS. 4-7. The identification of compounds may be based on the activity percentage 404 and the accuracy percentage 402. For instance, the identification of compounds may be of those having an activation required by the activity percentage 404 and the accuracy percentage 402.

As such, in step 902 a selection may be performed to identify compounds, e.g., active compounds, for further experimentation. As an example, compounds may be identified that have an activation above a reference defined by the activity percentage. Compounds may be identified that have an identified accuracy above a reference defined by the accuracy percentage. As such, assays of compounds or portions thereof may be identified for a secondary screen that have a Z′-factor of less than 0.5. The secondary screen may be performed in step 904 to reevaluate the selected compounds. In step 906, a structure-activity relation may be identified using the methods described herein, and in step 908, clinical trials may be performed on the compounds identified using methods described herein.

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

What is claimed is:

1. A method comprising:
receiving measured activity from a first assay, the first assay comprising wells containing compounds and first controls, wherein the measured activity is indicative of activation or inhibition by the compounds on a process;
determining an estimate of activity percentages of the compounds, wherein the estimate of the activity percentages of the compounds is based on a noise distribution and the noise distribution is based on the first controls;
receiving a first reference based on a required activity percentage of compounds for the first assay;
receiving a second reference based on a required accuracy percentage of the compounds identified as active according to the first reference; and
identifying active compounds of the first assay based on the first reference, the second reference, the measured activity, and the estimate of activity percentages.

2. The method of claim 1, further comprising:
preparing a second assay based on the active compounds of the first assay, wherein the second assay comprises second controls.

3. The method of claim 2, further comprising:
conducting an experiment on the second assay.

4. The method of claim 3, further comprising:
identifying active compounds of the second assay based on the first reference and the second reference, based on the measured activity of the experiment on the second assay.

5. The method of claim 1, wherein the noise distribution may be expressed as a Z′-factor.

6. The method of claim 5, wherein the Z′-factor is less than 0.5.

7. The method of claim 1, wherein the first reference and the second reference are based on a user input.

8. The method of claim 1, wherein the estimate of the activity percentages of the compounds is determined by steps further comprising:
predicting a power associated with the first assay based on a Type I error rate.

9. The method of claim 8, wherein the power is based on a Type II error rate.

10. The method of claim 1, further comprising:
adjusting error assumptions associated with the required activity percentage as non-linear errors.

11. The method of claim 10, wherein the error assumptions are based on a log-normal distribution, an inverse Gaussian distribution, a gamma distribution, or a skewed normal distribution or an error distribution measured based on the first controls or second controls.

12. The method of claim 1, wherein the estimate of the activity percentages of the compounds in the first assay are further based on a postulation of true activities of the compounds.

13. The method of claim 1, wherein the estimate of the activity percentages of the compounds in the first assay are further based on an estimate of true activities of the compounds.

14. The method of claim 13, wherein the estimate of the activity percentages of the compounds is based on steps comprising:
loading a set of measured activity percentages; and
reducing error between the set of measured activity percentages and the estimate of the activity percentages from the first assay.

15. The method of claim 14, wherein the set of measured activity percentages is based on a library of activity percentages.

16. The method of claim 14, wherein the estimate of the activity percentages of the compounds are further based on the noise distribution.

17. A method comprising:
synthesizing a compound of compounds, the compound selected by steps comprising:
receiving measured activity from a first assay, the first assay comprising wells containing one or more of the compounds and first controls, wherein the measured activity is indicative of activation or inhibition by the one or more of the compounds on a process;
determining activity percentages of the one or more of the compounds based on a noise distribution defined according to the first controls;
receiving a first reference based on a required activity percentage of one or more of the compounds determined in the first assay;
receiving a second reference based on a required accuracy percentage of the one or more of the compounds identified as active according to the first reference; and
identifying the compound in the first assay based on the first reference, the second reference, the measured activity, and the activity percentages.

18. The method of claim 17, wherein the noise distribution is a Z′-factor and the Z′-factor is less than 0.5.

19. A method comprising:
conducting an experiment on a second assay based on active compounds identified by steps comprising:
receiving measured activity from a first assay, the first assay comprising wells containing compounds and first controls, wherein the measured activity is indicative of activation or inhibition by the compounds on a process;
determining activity percentages of the compounds based on a noise distribution defined according to the first controls;
receiving a first reference based on a required activity percentage of compounds determined in the first assay;
receiving a second reference based on a required accuracy percentage of the compounds identified as active according to the first reference; and
identifying the active compounds in the first assay based on the first reference, the second reference, the measured activity, and the activity percentages.

20. The method of claim 19, wherein the noise distribution is a Z′-factor and the Z′-factor is less than 0.5.
